(19) United States
(12) Patent Application Publication
(10) Pub. No.: US 2010/0024098 A1
(43) Pub. Date: Feb. 4, 2010

(54) Swimming goggles

(76) Inventor: Herman Chiang, Chung-Ho City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/222,244

(22) Filed: Aug. 6, 2008

(30) Foreign Application Priority Data
Jul. 29, 2008 (TW) 97213426

(51) Int. Cl. A61F 9/02

(52) U.S. Cl. 2/428; 2/445; 2/442

(57) ABSTRACT

Swimming goggles include a left bracket, a right bracket, a hard nose support between the left bracket and the right bracket, lenses respectively assembled on the left bracket and the right bracket, soft integral assembly of frame, nose bridge and pad, and a head strap. The left bracket and the right bracket are made of rigid material, and form a plurality of latch portions and define injection orifices thereon. Strap assembling blocks are formed on outward sides of the left bracket and the right bracket for assembling with the head strap. Through holes are defined in the lenses for corresponding to the injection orifices. Soft integral frame, nose bridge and pad is made in such a way that during shaping, the left bracket, the right bracket and the hard nose support are covered along peripherals of the lenses with the strap assembling blocks being exposed outward.

### BACKGROUND OF THE INVENTION

1. Field of the Invention

The present invention relates to swimming goggles, and particularly to swimming goggles which have frames, nose bridge and pads integrated together.

2. Related Art

Swimming goggles, which have frames, nose bridge and pads integrated together, normally concern with preventing water leakage arising from deformed frames and improperly assembled lenses. At the same time, comfortable touch to face is necessary for users when swimming goggles are worn. Conventional swimming goggles with integrated frames, nose bridge and pads, therefore, adopt relatively soft material, and strengthen the nose bridge. For instance, the nose bridge has a thick middle part and thin side parts. Such conventional swimming goggles are probably deformed, and can not touch users' faces comfortably. Lenses of such conventional swimming goggles must be mounted by implantation, that is, the lenses are placed on a shaped mold in advance for integral shaping.

However, such conventional swimming goggles are made of relatively soft material, therefore structure design thereof becomes more difficult, lest deformation of frames and mar of connection between the lenses and the frames, resulting in water leakage. Besides, the reinforced nose bridge has worse flexibility, and to some extent, cannot meet users' nose profiles. That is, if a user has relatively high nose profile, the nose bridge may press against the user's nose so that the user feels uncomfortable.

### SUMMARY OF THE INVENTION

To overcome the shortcomings, an object of the present invention is to provide swimming goggles with integrated frames, nose bridge and pad, which are worn comfortably and meet users with different nose profiles, and which have undeformed frames and fixed lenses for preventing leakage.

The swimming goggles comprise a left bracket, a right bracket, a hard nose support, lenses being respectively assembled on the left bracket and the right bracket, a soft integral assembly of frame, nose bridge and pad, and a head strap. The left bracket and the right bracket are made of rigid material, and form a plurality of latch portions and define injection orifices thereon. Strap assembling blocks are formed on outward sides of the left bracket and the right bracket. The hard nose support is assembled between the left bracket and the right bracket, and has appropriate flexibility. Lenses are respectively assembled between the left bracket and the right bracket. Lock portions are formed on peripherals of the lenses. Through holes are defined in the lenses for corresponding to the injection orifices. Soft integral frame, nose bridge and pad is made in such a way that during shaping, the left bracket, the right bracket and the hard nose support are covered along peripherals of the lenses with the strap assembling blocks being exposed outward. The head strap is assembled on the strap assembling blocks.

The latch portions of the left bracket and the right bracket are protuberances, and wherein the lock portions are assembling holes for assembling with the protuberances.

The hard nose support extends unitarily from inward sides of the left bracket and the right bracket, and comprises a narrow center portion, and slanted ribs extending from both sides of the center portion for providing flexibility and enough side strength against pulling force when the swimming goggles are worn.

### BRIEF DESCRIPTION OF THE DRAWINGS

FIG. 1 is an exploded view of swimming goggles of the present invention.

FIGS. 2 is an assembled view of the swimming goggles of FIG. 1.

FIG. 3 is a front view of the swimming goggles of FIG. 2.

FIG. 4 is a cross-sectional view taken along the line 4-4 in FIG. 3.

FIG. 5 is a cross-sectional view taken along the line 5-5 in FIG. 3.

FIG. 6 is a cross-sectional view taken along the line 6-6 in FIG. 3.

### DESCRIPTION OF THE PREFERRED EMBODIMENTS

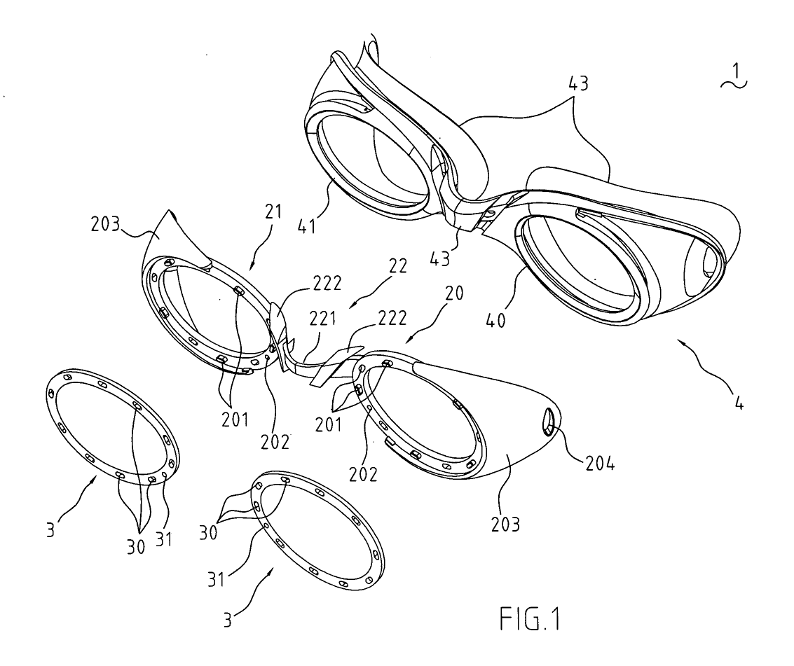

With reference to FIG. 1, integral type swimming goggles 1 of the present invention comprise left bracket 20, right bracket 21, a hard nose support 22, lenses 3, a soft integral assembly of frame, nose bridge and pad 4, and a head strap (not shown). The left bracket 20 and the right bracket 21 are made of rigid material, for example polypropylene (PP). A plurality of latch portions are formed on the left bracket 20 and the right bracket 21, and in this embodiment include protuberances 201. Injection orifices 202 are defined in the left bracket 20 and the right bracket 21. Strap assembling blocks 203 are formed on outward sides of the left bracket 20 and the right bracket 21. In this embodiment, the strap assembling blocks 203 respectively extend from middle of outward sides of the left bracket 20 and the right bracket 21, and respectively form arc surfaces. Each strap assembling block 203 defines a strap hole 204 near an outward edge of the arc surface for assembling the head strap. Color-change power may be mixed into the material of the strap assembling blocks 203. As a consequence, the strap assembling blocks 203 can take on different colors with different ray luminance, making users appear variable colors indoor or outdoor.

The hard nose support 22 extends unitarily from inward sides of the left bracket 20 and the right bracket 21, and comprises a narrow center portion 221, and slanted ribs 222 extending from both sides of the center portion 221. So the hard nose support 22 has enough flexibility, and enough strength against pulling force when the swimming goggles are worn.

The lenses 3 are respectively assembled on the left bracket 20 and the right bracket 21. Lock portions are formed on peripherals of the lenses 3. In this embodiment, lock portions include assembling holes 30 for assembling with the protuberances 201. Through holes 31 are defined in the lenses 3 for corresponding to the injection orifices 202 of the left bracket 20 and the right bracket 21.

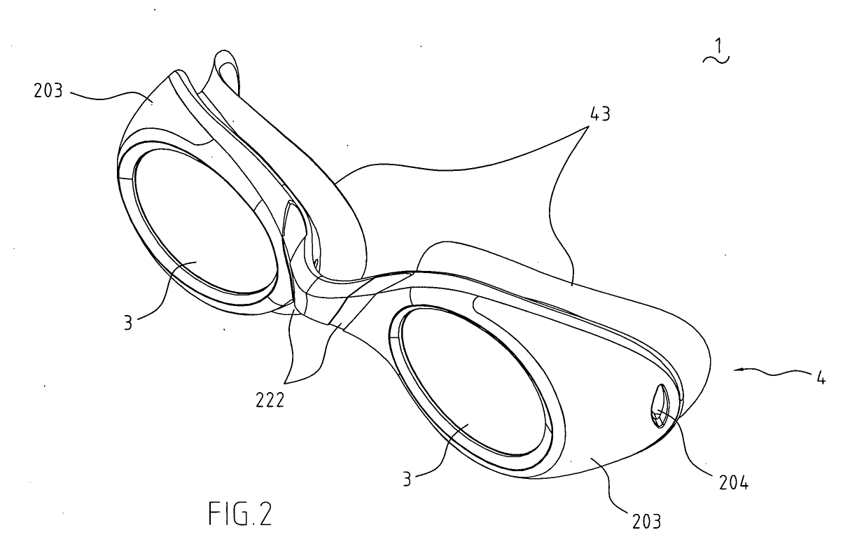

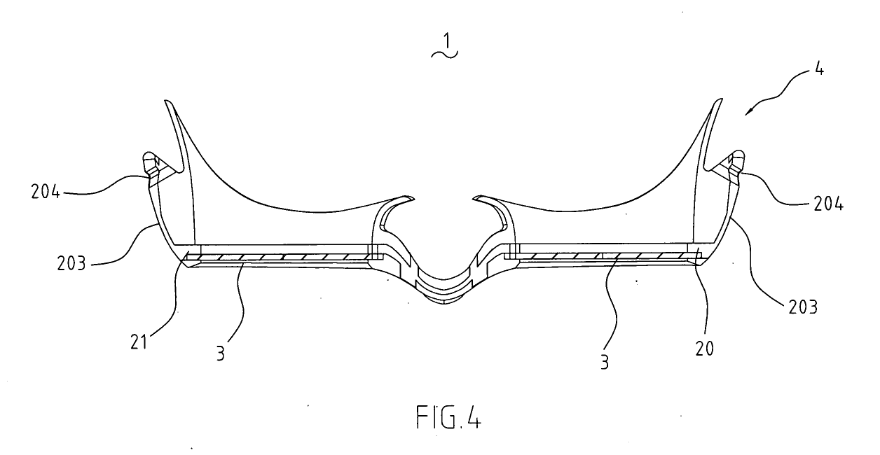

The soft integral assembly of frame, nose bridge and pad 4 is made of Thermal Plastic Rubber (TPR), and comprises a left frame 40, a right frame 41, a nose bridge 42, and pads 43. Firstly, the assembling holes 30 and the protuberances 201 of the left bracket 20 and the right bracket 21 lock with each other, and the lenses 3 are preliminarily positioned. During shaping, the left bracket 20, the right bracket 21 and the hard nose support 22 cover peripherals of the lenses 3. The strap assembling blocks 203 and the slanted ribs 222 of the hard nose support 22 are exposed outward. The shaping plastic passes the through holes 31 and the injection orifices 202, reliably enveloping peripherals of the lenses 3, the left bracket 20 and the right bracket 21, and the strap holes 204 being exposed out for assembling with the head strap, as shown in FIGS. 2 and 4.

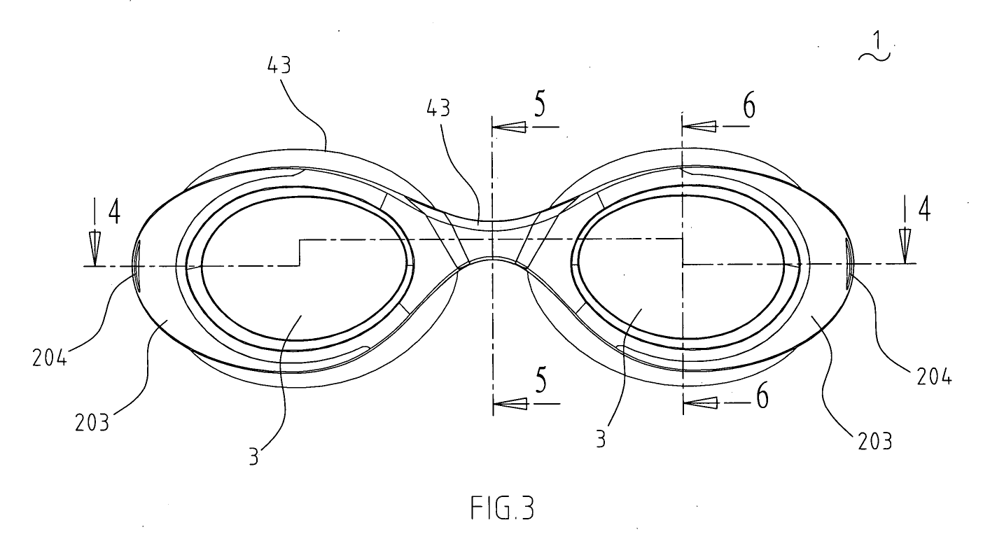

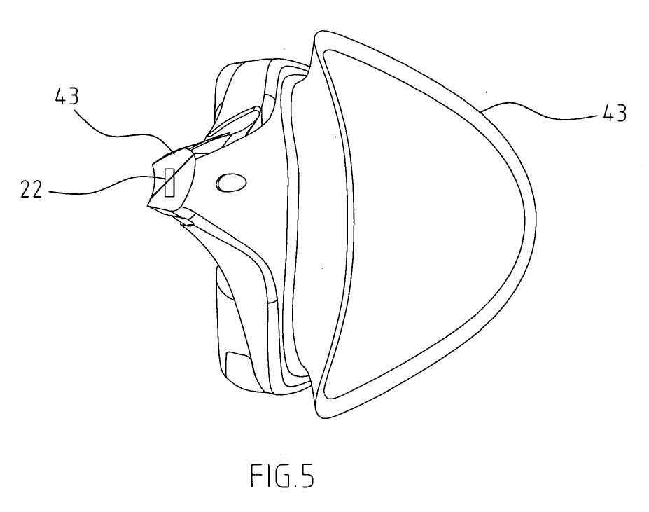

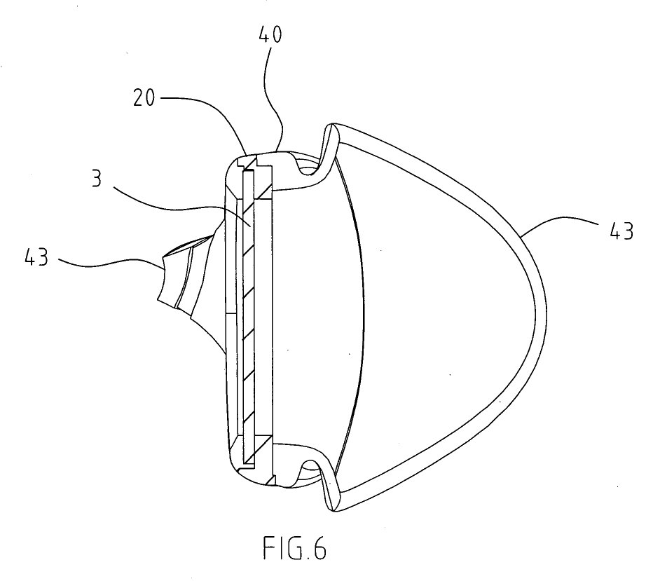

Further referring to FIGS. 3-6, the lenses 3 are assembled on the left bracket 20 and the right bracket 21 and are then covered. The head strap is assembled on the strap holes 204 of the strap assembling blocks 203. Thus, when the swimming goggles are worn, the pulling force of the head strap will not result in deformation of the soft left frame 40 and right frame 41. Also, the soft nose bridge 43 envelops the hard nose support 22 for providing appropriate flexibility thereby fitting for users with different nose profiles.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. Swimming goggles comprising:
a left bracket and a right bracket being made of rigid material, forming a plurality of latch portions and defining injection orifices thereon, strap assembling blocks being formed on outward sides of the left bracket and the right bracket;
a hard nose support being assembled between the left bracket and the right bracket, and having appropriate flexibility;
lenses being respectively assembled on the left bracket and the right bracket, lock portions being formed on peripherals of the lenses for corresponding to the latch portions, through holes being defined in the lenses for corresponding to the injection orifices;
soft integral assembly of frame, nose bridge and pad, being made in such a way that during shaping, the left bracket, the right bracket and the hard nose support are covered along peripherals of the lenses with the strap assembling blocks being exposed outward; and
a head strap assembled on the strap assembling blocks.

2. The swimming goggles as claimed in claim 1, wherein the latch portions of the left bracket and the right bracket include protuberances, and wherein the lock portions include assembling holes for assembling with the protuberances.

3. The swimming goggles as claimed in claim 2, wherein the hard nose support extends unitarily from inward sides of the left bracket and the right bracket, and comprises a narrow center portion and slanted ribs extending from both sides of the center portion for providing flexibility and enough side strength against pulling force when being worn.

4. The swimming goggles as claimed in claim 3, wherein the left bracket, the right bracket and the hard nose support are covered, the slanted ribs of the hard nose support being exposed out.

5. The swimming goggles as claimed in claim 4, wherein the strap assembling blocks respectively extend from middle of outward sides of the left bracket and the right bracket for respectively form arc surfaces, each strap assembling block defining a strap hole near an outward edge of the arc surface for assembling the head strap.

6. The swimming goggles as claimed in claim 5, wherein color-change power is mixed into the material of the strap assembling blocks so that the strap assembling blocks take on different colors with different ray luminance.
